(19) United States
(12) Patent Application Publication
(10) Pub. No.: US 2016/0367262 A1
(43) Pub. Date: Dec. 22, 2016

(54) Tourniquet and Windlass Assembly and Method

(71) Applicants: Samuel E. Burke, Stafford, VA (US); Justin Cuomo, Woodbridge, VA (US)

(72) Inventors: Samuel E. Burke, Stafford, VA (US); Justin Cuomo, Woodbridge, VA (US)

(21) Appl. No.: 14/742,825

(22) Filed: Jun. 18, 2015

(51) Int. Cl. A61B 17/132

(52) U.S. Cl. CPC A61B 17/1322; A61B 17/1327; A61B 2017/12004

(57) ABSTRACT

A tourniquet and windlass marking assembly for use in treating a bleeding wound includes a tourniquet body having an elongate and self-adhering main strap configured to extend around a limb of a person having a bleeding wound. A windlass strap is coupled to the main strap and configured to tighten the tourniquet body about the bleeding wound. A time stamp member is coupled to the tourniquet body and has a surface configured to receive indicia thereon. A windlass shaft is operatively coupled to the windlass strap and configured to tighten the main strap when the windlass shaft is rotated. Together, the lower end, upper end, and upper end of the windlass shaft define an interior area selectively accessible through the open upper end. A marking device is removably situated in the interior area of the windlass for selectively applying indicia to the time stamp member.

### BACKGROUND OF THE INVENTION

This invention relates generally to a wound treatment device and, more particularly, to a tourniquet and windlass assembly having a marking device stored within the windlass shaft for quickly and easily recording in real time a time of application of a tourniquet to a bleeding wound.

A tourniquet is a constricting or compressing device in the nature of a bandage that is used to control venous and arterial circulation to an extremity for a period of time. Pressure is applied circumferentially upon the skin and underlying tissues of a limb and transferred to the walls of vessels, causing them to become temporarily occluded. It is generally used as a tool for a medical professional to stem the flow of traumatic bleeding, especially by military medics. The tourniquet is usually applied when the patient is in a life-threatening state as a result of continuous bleeding. It is important not to excessively tighten the tourniquet or to leave the tourniquet in place for too long as that may result in permanent damage to the limb or even result in an amputation.

Various devices have been proposed for quickly and efficiently applying a tourniquet to a person's arm or leg above the point of bleeding. Although presumably effective for their intended purposes, the existing tourniquet products do not provide a convenient means for a medical professional to record a specific time in which the tourniquet was applied and to record the time in a manner and location so that it will be seen by personnel at a subsequent medical facility.

Therefore, it would be desirable to have a tourniquet and windlass marking assembly that provides a marking device stored in a windlass shaft that is selectively used to record a time at which a tourniquet was applied to the limb of a person. Further, it would be desirable to have a tourniquet and windlass marking assembly that is threadably held in or removed from the windlass shaft and configured to mark a time on a time stamp member of the tourniquet.

### SUMMARY OF THE INVENTION

A tourniquet and windlass marking assembly for use in treating a bleeding wound according to the present invention includes a tourniquet body having an elongate and self-adhering main strap configured to extend around a limb of a person having a bleeding wound. A windlass strap is coupled to the main strap and configured to selectively and incrementally tighten the tourniquet body about the bleeding wound. A time stamp member is coupled to the tourniquet body and has a surface configured to receive indicia thereon.

A windlass shaft includes opposed upper and lower ends and a continuous side wall extending therebetween, the windlass shaft being operatively coupled to the windlass strap and configured such that the main strap is tightened when the windlass shaft is rotated relative to the windlass strap. The lower end of the windlass shaft is closed, the upper end is open, and the side wall of the windlass shaft defines an interior area selectively accessible through the open upper end. A marking device is removably situated in the interior area of the windlass for selectively applying indicia to the time stamp member.

Therefore, a general object of this invention is to provide a tourniquet and windlass marking assembly having a marking instrument stored within a windlass shaft of a tourniquet.

Another object of this invention is to provide a tourniquet and windlass marking assembly, as aforesaid, in which the marking device is threadably coupled to the windlass shaft when not in use.

Still another object of this invention is to provide a tourniquet and windlass marking assembly, as aforesaid, in which the marking device is operable to mark a time indicia on a time stamp member of a tourniquet.

Yet another object of this invention is to provide a tourniquet and windlass marking assembly, as aforesaid, that is easy to use and cost effective to manufacture.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a tourniquet and windlass marking assembly according to a preferred embodiment of the present invention illustrated in a tightened configuration;

FIG. 2a is a perspective view of the assembly as in FIG. 1, illustrated in a start configuration;

FIG. 2b is another perspective view of the assembly as in FIG. 2a, illustrated in a partially tightened configuration;

FIG. 2c is another perspective view of the assembly as in FIG. 2a, illustrated in a further tightened configuration;

FIG. 2d is another perspective view of the assembly as in FIG. 2a, illustrated in a fully tightened configuration;

FIG. 3a is an exploded view of a windlass shaft and marking device of the assembly as in FIG. 1;

FIG. 3b is a further exploded view of the windlass shaft and marking device as in FIG. 3a;

FIG. 4a is a side view of the windlass shaft removed from the assembly as in FIG. 2b;

FIG. 4b is a sectional view taken along line 4b-4b of FIG. 4a; and

FIG. 4c is an isolated view on an enlarged scale taken from FIG. 4b.

### DESCRIPTION OF THE PREFERRED EMBODIMENT

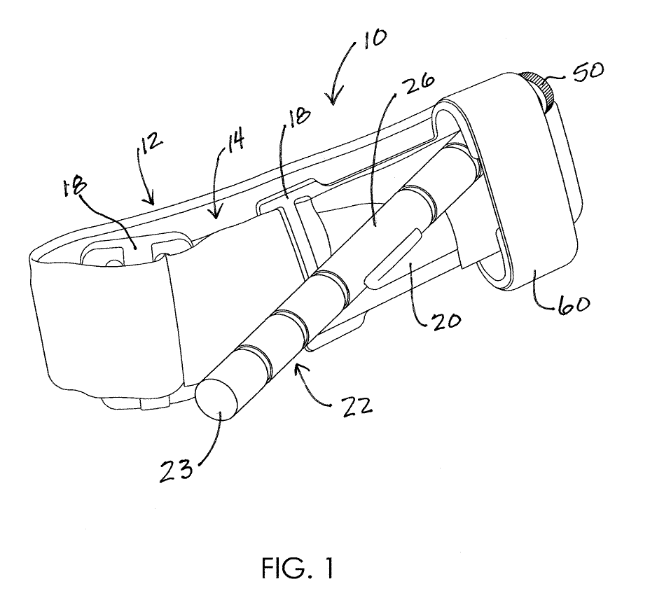

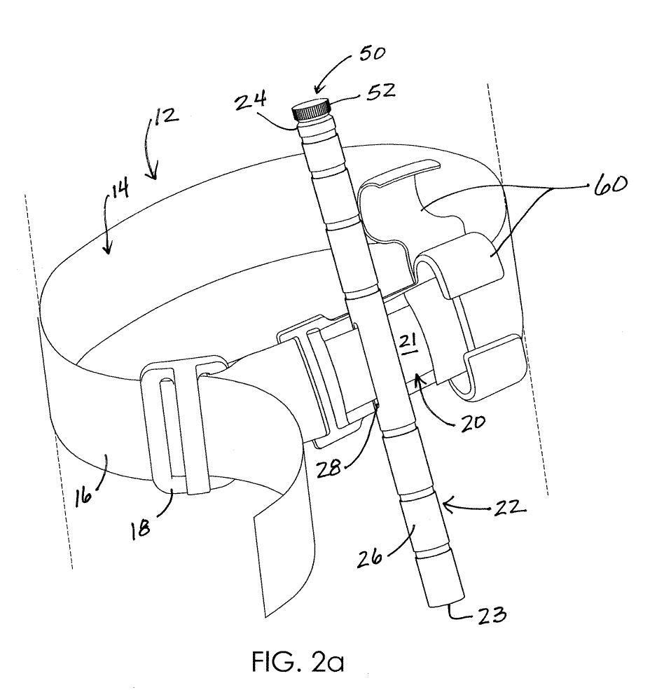

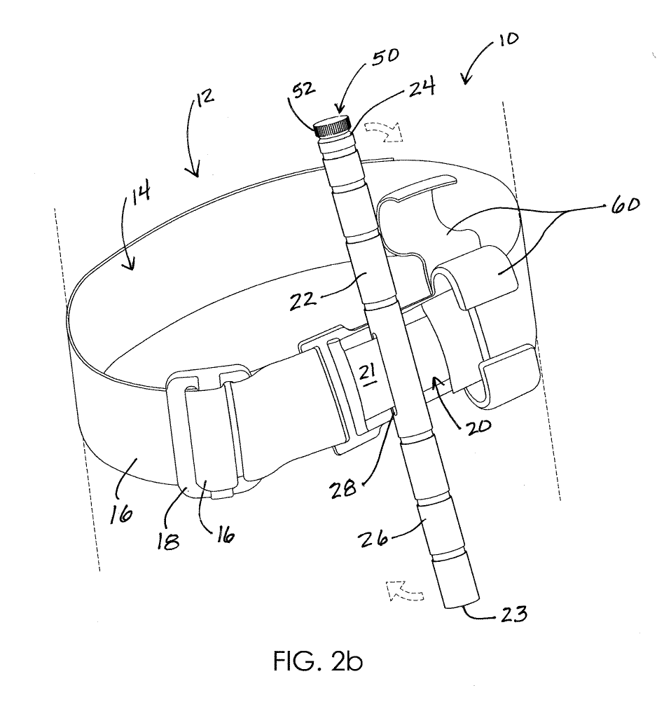

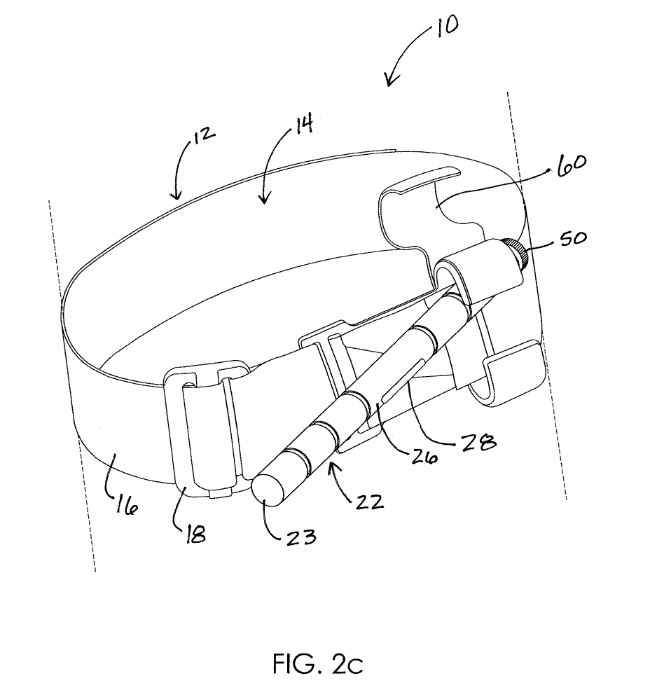

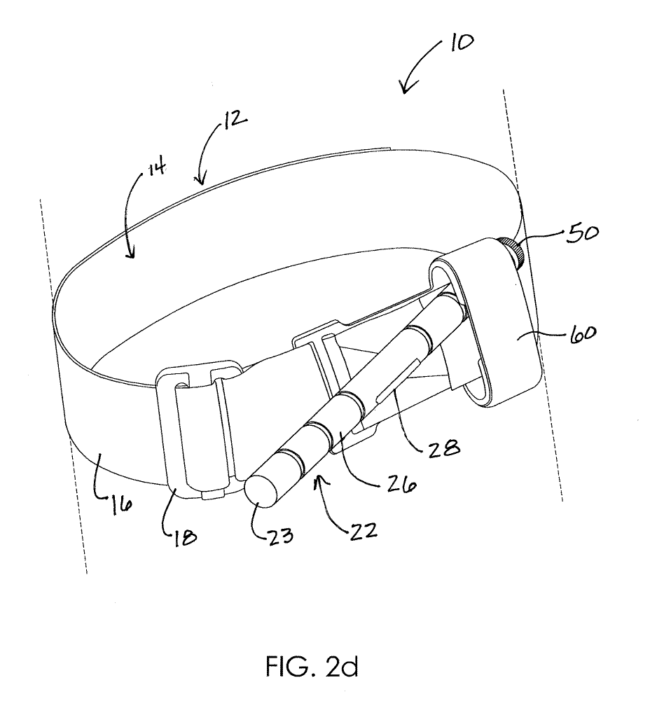

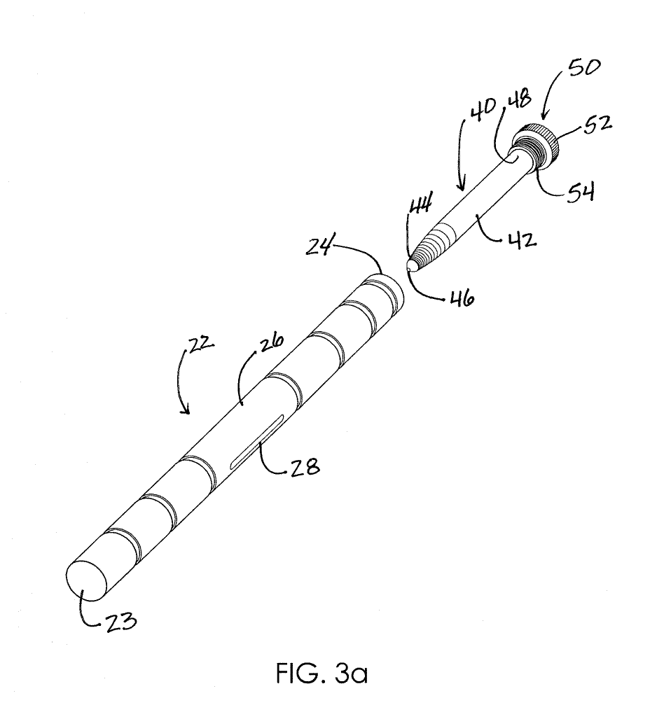

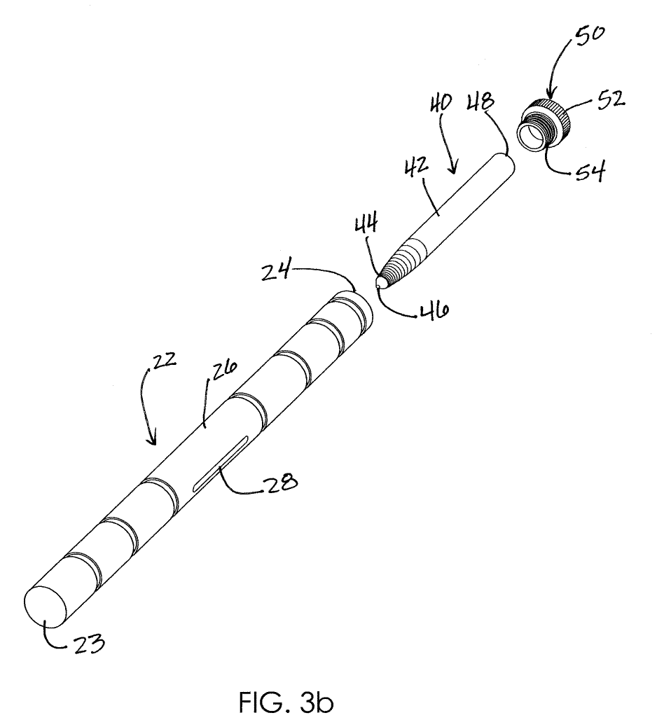

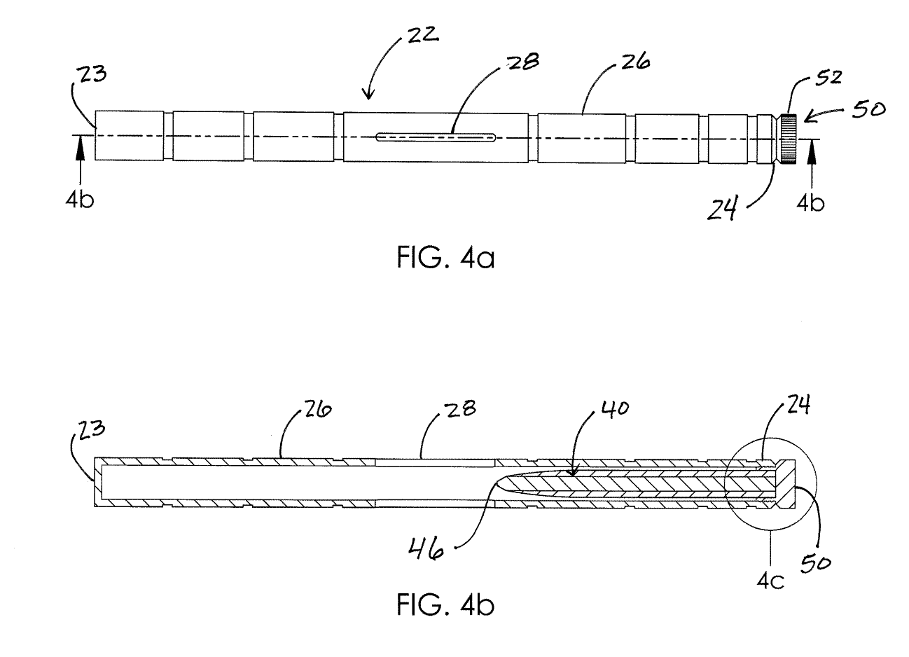

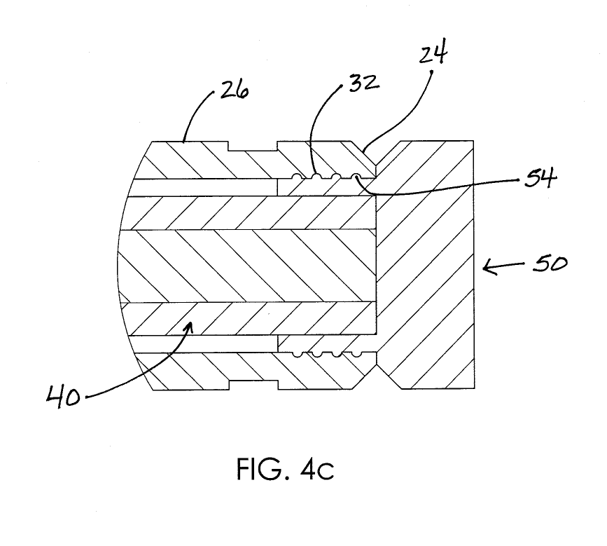

A tourniquet and windlass marking assembly for use in treating a bleeding wound according to a preferred embodiment of the present invention will now be described in detail with reference to FIGS. 1 to 4c of the accompanying drawings. The tourniquet and windlass marking assembly 10 includes a tourniquet body 14, a windlass shaft 22 for tightening the tourniquet body 14 about the limb of a patient having a bleeding wound, and a marking device 40 selectively received in an interior area defined by the windlass shaft 22.

The tourniquet to be used in the present invention includes a tourniquet body 14 having an elongate and generally flexible main strap 16 that is able to extend around the leg or arm of an injured person or patient. The main strap 16 may include appropriately positioned hook and loop fasteners so that the main strap 16 can be doubled back and attached to itself once a person's limb has been encircled. The tourniquet body 14 may include a buckle 18 through which the main strap 16 may be extended to facilitate the folding back on itself described above as well as to enable the main strap 16 to be incrementally tightened about the patient's limb. In this manner, the main strap 16 may be referred to as being self-adhering to itself when secured to a person's arm or leg.

The tourniquet includes a windlass strap 20 coupled to or integrally formed to the main strap 16 and is arranged such that the main strap 16 is tightened when the windlass strap 20 is twisted as will be described in more detail below. The windlass strap 20 may include opposed first and second ends coupled to the main strap 16 and include a middle section 21 that extends between the opposed ends.

The tourniquet and windlass marking assembly 10 includes a windlass shaft 22 coupled to the windlass strap 20 and is useful in tightening the tourniquet body 14 to a limb of a person being treated for a bleeding wound. The windlass shaft 22 is in the form of a rod having a lower end 23, an opposed upper end 24, and a continuous side wall 26 extending between the opposed ends. The windlass shaft 22 has an elongate configuration so as to provide leverage and ease of grip to a user in that the windlass shaft 22 needs to be twisted multiple times to tighten about a person's limb so as to stop the flow of blood to the wound. The windlass shaft 22 defines a slot 28 or channel through which the middle section 21 of the windlass strap 20 extends. Therefore, the windlass strap 20 is twisted when the windlass shaft 22 is twisted or rotated, the twisting of the windlass strap 20 essentially pulling portions of the main strap 16 together to tighten around the patient's arm or leg.

The side wall 26 of the windlass shaft 22 defines an interior area 30. In other words, the windlass shaft 22 defines a cavity or is completely hollow. The lower end 23 of the windlass shaft 22 is closed whereas the upper end 24 is open. Together, therefore, the windlass shaft 22 provides a sleeve that may be accessed through the open upper end 24.

Further, the marking device 40 according to the present invention includes a diameter that is smaller than an inner diameter of the interior area 30 of the windlass shaft so as to be selectively received therein. More particularly, the marking device 40 includes a body portion 42 having a cylindrical configuration and a diameter that allows the body portion 42 to be slidably received through the upper end 24 into the interior area 30. The marking device 40 includes a first end 44 having a writing tip 46 and a second end 48 having a cap 50. The cap 50 includes an outer diameter that is greater than a diameter of the open upper end 24 so as to block access to the interior area 30 when the marking device 40 is inserted therein. Preferably, the marking device 40 is a grease pencil in that such a writing instrument is capable of writing on less than clean surfaces, in the rain, etc.

In an embodiment, the windlass shaft 22 includes a first threaded section 32 positioned on an inner surface adjacent the upper end 24. Correspondingly, the body portion 42 of the marking device 40 includes a second threaded section 54 adjacent (below) the cap 50 that is configured to selectively and threadably receive the first threaded section 32 in a threadably secured arrangement. An outer edge 52 of the cap 50 may include a roughened grip surface that enhances a user's ability to grip the cap 50 and turn it to remove the marking device 40 from the interior area. Further, the cap 50 may include a color indicia, such as tan, that makes it easier for a user to perceive which end of the windlass shaft 22 is the cap 50 of the marking device 40 and not the closed lower end 23. Still further, the cap 50 itself may be removably coupled to the body portion 42 of the marking device 40 such that the body portion 42 is interchangeable and replaceable.

The tourniquet 12 includes a time stamp member 60 coupled to the tourniquet body 14. The time stamp member 60 may be constructed of a flexible material and include a surface capable of receiving indicia from a writing instrument. The stamp member 60 may also include hook and loop fasteners such that the time stamp member 60 may be self-adhering to the tourniquet body 14 and used, for example, to selectively hold the windlass shaft 22 at a fixed position after being twisted. The surface of the time stamp member 60 may include a white color so that written markings are clearly viewable. The stamp member 60 may include the word “TIME” imprinted thereon to prompt or remind a first responder to write the time of tourniquet application on the surface.

In use, a “first responder” to a location where a person is experiencing a bleeding wound may prepare to stop the bleeding and move the patient to a medical facility by applying the tourniquet and marking assembly 10. First, the responder should locate the specific location that is bleeding. Then, the tourniquet and windlass marking assembly 10 should be positioned about 2 to 3 inches above the bleeding wound. More particularly, the tourniquet body 14 should be wrapped the patient's arm or leg and secured back upon itself as described above. Then, the windlass shaft 22 is twisted using the windlass shaft 22 such that the windlass strap 20 tightens the main strap 16 until the bleeding stops. Then, the time stamp member 60 may be adhered over the top of the windlass shaft 22 so that it does not turn in reverse to loosen its grasp on the patient's bleeding limb. Then, the cap 50 of the marking device 40 is twisted and the marking device 40 is threadably removed from the interior area of the windlass shaft 22. Finally, a current time indicative of when the tourniquet is being applied may be written on the surface of the time stamp member 60 for future reference, such as when the patient is checked into a medical facility. After use, the marking device 40 may be returned into the interior area of the windlass shaft for storage or until another use is necessary.

It is understood that while certain forms of this invention have been illustrated and described, it is not limited thereto except insofar as such limitations are included in the following claims and allowable functional equivalents thereof.

I claim:

1. A tourniquet and windlass marking assembly for use in treating a bleeding wound, comprising:
a tourniquet body having an elongate and self-adhering main strap configured to extend around a limb of a person having a bleeding wound;
a windlass strap coupled to said main strap and configured to selectively tighten said tourniquet body about the bleeding wound;
a time stamp member coupled to said tourniquet body and having a surface configured to receive indicia thereon;
a windlass shaft having opposed upper and lower ends and a continuous side wall extending therebetween, said windlass shaft being operatively coupled to said windlass strap and configured such that said main strap is tightened when said windlass shaft is rotated relative to said windlass strap;
wherein said lower end of said windlass shaft is closed, said upper end is open, and said side wall of said windlass shaft defines an interior area selectively accessible through said open upper end; and
a marking device removably situated in said interior area of said windlass for selectively applying indicia to said time stamp member.

2. The tourniquet and windlass marking assembly as in claim 1, wherein said marking device includes:
a body portion having an outer diameter that is smaller than an inner diameter of said interior area so as to be selectively received therein;
a first end having a writing tip; and
a second end opposed from said first end having a cap, said cap having an outer diameter greater than a diameter of said open second end so as to block access to said interior area.

3. The tourniquet and windlass marking assembly as in claim 2, wherein:
said windlass shaft includes a first threaded section positioned on an interior surface adjacent said upper end; and
said body portion of said marking device includes a second threaded section adjacent said cap, said first threaded section configured to selectively receive said second threaded section so as to secure said marking device in said interior area of said windlass shaft.

4. The tourniquet and windlass marking assembly as in claim 1, wherein:
said windlass strap includes a first end and a second end coupled to said main strap of said tourniquet body;
said windlass shaft defines a slot situated between said upper and lower ends, said slot selectively receiving a middle portion of said windlass strap that extends between said first and second ends therethrough.

5. The tourniquet and windlass marking assembly as in claim 2, wherein said cap of said marking device includes an outer edge having a grip surface that enhances a user's grasp when threadably inserting or removing said marking device from said interior area.

6. The tourniquet and windlass marking assembly as in claim 2, wherein said cap includes a color indicia so as to be easily noticed and contrasted with said closed end of said windlass shaft.

7. The tourniquet and windlass marking assembly as in claim 1, wherein said tourniquet body includes a buckle about which a free end of said main strap is selectively extended such that said main strap is tightened upon a twisting of said windlass strap.

8. The tourniquet and windlass marking assembly as in claim 1, wherein said marking device is a grease pencil capable of writing on wet or dirty surfaces.

9. The tourniquet and windlass marking assembly as in claim 2, wherein said cap is removably coupled to said second end of said marking device, whereby said marking device is replaceable.

10. The tourniquet and windlass marking assembly as in claim 1, wherein:
said marking device includes:
a body portion having an outer diameter that is smaller than an inner diameter of said interior area so as to be selectively received therein;
a first end having a writing tip;
a second end opposed from said first end having a cap, said cap having an outer diameter greater than a diameter of said open second end so as to block access to said interior area;
said windlass shaft includes a first threaded section positioned on an interior surface adjacent said upper end; and
said body portion of said marking device includes a second threaded section adjacent said cap, said first threaded section configured to selectively receive said second threaded section so as to secure said marking device in said interior area of said windlass shaft.

11. A method for treating a bleeding wound, comprising:
locating the location of a bleeding wound;
securing a tourniquet and windlass marking assembly about 2 to 3 inches above the bleeding wound, said tourniquet and windlass marking assembly, comprising:
a tourniquet body having an elongate and self-adhering main strap configured to extend around a limb of a person having a bleeding wound;
a windlass strap coupled to said main strap and configured to selectively tighten said tourniquet body about the bleeding wound;
a time stamp member coupled to said tourniquet body and having a surface configured to receive indicia thereon;
a windlass shaft having opposed upper and lower ends and a continuous side wall extending therebetween, said windlass shaft being operatively coupled to said windlass strap and configured such that said main strap is tightened when said windlass shaft is rotated relative to said windlass strap;
wherein said lower end of said windlass shaft is closed, said upper end is open, and said side wall of said windlass shaft defines an interior area selectively accessible through said open upper end; and
providing a marking device removably situated in said interior area of said windlass for selectively applying indicia to said time stamp member; and
marking a time on said surface of said time stamp member that is indicative of when said tourniquet body was applied to the bleeding wound.

12. The method for treating a bleeding wound as in claim 11, wherein said marking device includes:
a body portion having an outer diameter that is smaller than an inner diameter of said interior area so as to be selectively received therein;
a first end having a writing tip; and
a second end opposed from said first end having a cap, said cap having an outer diameter greater than a diameter of said open second end so as to block access to said interior area.

13. The method for treating a bleeding wound as in claim 12, wherein:
said windlass shaft includes a first threaded section positioned on an interior surface adjacent said upper end; and
said body portion of said marking device includes a second threaded section adjacent said cap, said first threaded section configured to selectively receive said second threaded section so as to secure said marking device in said interior area of said windlass shaft.

14. The method for treating a bleeding wound as in claim 13, further comprising threadably releasing said cap from said windlass shaft and removing said marking device from said interior area.

15. The method for treating a bleeding wound as in claim 12, further comprising writing a time on said time stamp using said writing tip of said marking device.

16. The method for treating a bleeding wound as in claim 12, wherein said cap of said marking device includes an outer radial surface having a grip surface that enhances a user's grasp when threadably inserting or removing said marking device from said interior area.

17. The method for treating a bleeding wound as in claim 12, wherein said cap includes a color indicia so as to be easily noticed and contrasted with said closed end of said windlass shaft.

18. The method for treating a bleeding wound as in claim 11, wherein said tourniquet body includes a buckle about which a free end of said main strap is selectively extended such that said main strap is tightened upon a twisting of said windlass strap.

19. The method for treating a bleeding wound as in claim 11, wherein said marking device is a grease pencil capable of writing on wet or dirty surfaces.

20. The method for treating a bleeding wound as in claim 12, wherein said cap is removably coupled to said second end of said marking device, whereby said marking device is replaceable.
